(19) United States
(12) Patent Application Publication
(10) Pub. No.: US 2024/0268846 A1
(43) Pub. Date: Aug. 15, 2024

(54) MEDICAL INSTRUMENT AND MEDICAL INSTRUMENT SET

(71) Applicant: Aesculap AG, Tuttlingen (DE)

(72) Inventors: Sven Barthelmes, Emmingen-Liptingen (DE); Pedro Morales, Tuttlingen (DE)

(21) Appl. No.: 18/610,831

(22) Filed: Mar. 20, 2024

Related U.S. Application Data
(63) Continuation of application No. PCT/EP2022/076377, filed on Sep. 22, 2022.

(30) Foreign Application Priority Data
Sep. 24, 2021 (DE) 10 2021 124 786.1

(51) Int. Cl. A61B 17/28

(52) U.S. Cl. CPC A61B 17/2816

(57) ABSTRACT

A medical instrument has a first instrument part and second instrument part. The first and second instrument parts are mounted to one another, pivotable about a pivot axis, and coupled to one another with a connecting element. The connecting element has a head accommodated in a head recess on the first instrument part. The head recess is open pointing from the first instrument part in a direction away from the second instrument part. The head has a circumferential, self-contained first annular face pointing away from the longitudinal axis. The head recess has a circumferential, self-contained second annular face pointing toward the longitudinal axis. The first and second annular faces are separated by an annular gap. A sealing element is arranged in the annular gap, in contact with the first and second annular faces. A medical instrument set includes medical instruments differing in shape, type, and/or size.

### CROSS-REFERENCE TO RELATED APPLICATIONS

This application is a continuation of International Application No. PCT/EP2022/076377, filed on Sep. 22, 2022, and claims priority to German Application No. 10 2021 124 786.1, filed on Sep. 24, 2021. The contents of International Application No. PCT/EP2022/076377 and German Application No. 10 2021 124 786.1 are incorporated by reference herein in their entireties.

### FIELD

The present disclosure relates to a medical instrument comprising a first instrument part and at least one second instrument part, wherein the at least one first instrument part and the at least one second instrument part are mounted to one another so as to be pivotable about a pivot axis and are coupled to one another with a connecting element, wherein a longitudinal axis of the connecting element defines the pivot axis, wherein the connecting element has a head, which is at least partially accommodated in a head recess on the first instrument part, wherein the head recess is open pointing from the first instrument part in a direction away from the at least one second instrument part, wherein the head has at least one circumferential, self-contained first annular face pointing away from the longitudinal axis, wherein the head recess has at least one circumferential, self-contained second annular face pointing in the direction toward the longitudinal axis, and wherein the at least one first annular face and the at least one second annular face are separated from one another in the radial direction relative to the longitudinal axis by an annular gap.

Furthermore, the present disclosure relates to a medical instrument set comprising at least two medical instruments differing in shape, type, and/or size.

### BACKGROUND

Medical instruments of the kind described at the outset are known in a variety of forms, for example they can be configured as scissors, clamps, or needle holders.

A problem with such medical instruments is, in particular, that tissue remnants, dirt, and body fluid can collect and adhere in the annular gap between the head of the connecting element and the head recess. This does not matter for instruments intended for single use. However, if such medical instruments are used multiple times, they must be reprocessed. They are typically washed in a dishwasher and then subjected to a superheated steam sterilization process. Due to the annular gap, which must necessarily be provided in order to be able to connect the instrument parts to one another at all, there is a risk that contaminants will not be completely removed from the annular gap during the processing of the instruments.

### SUMMARY

It is therefore an object of the present disclosure to improve a medical instrument and a medical instrument set of the kind described at the outset so that they are easy to clean.

This object is achieved, in accordance with the disclosure, in a medical instrument of the kind described at the outset in that a sealing element is arranged in the annular gap and in that the sealing element is in contact with the at least one first annular face and with the at least one second annular face.

Equipping a medical instrument as proposed with a sealing element, which is arranged in the annular gap and contacts both annular faces, makes it possible, in particular, to seal the annular gap and, for example, to fill it completely. Thus, it can be prevented that body fluids or rinsing liquids used during an operation can seep into the annular gap and, in particular, between mutually facing side faces of the abutting instrument parts due to capillary action. Thus, it is possible with the sealing element to seal this annular gap, to fill it at least partially, in particular completely, and to thus prevent the ingress of contaminants.

In order to achieve a permanently good seal of the annular gap, it is favorable if the sealing element and the annular gap form an interference fit. Thus, the sealing element is preferably larger than the annular gap before it is inserted into said gap.

It is favorable if the sealing element in a basic position, in which it is out of engagement with the first and/or the second annular face, defines a first sealing element shape, if the sealing element in an assembly position, in which it is arranged in the annular gap, defines a second sealing element shape, and if the second sealing element shape and the first sealing element shape differ from one another. Such a sealing element enables, in particular, a permanent and reliable sealing or filling of the annular gap. In particular, for this purpose, the sealing element may be elastically and/or plastically deformable. In particular, manufacturing tolerances, both for the annular gap and for the sealing element, can thus be compensated in a simple manner. In particular, such a sealing element allows to change a dimension thereof between the mutually facing annular faces, so as to fill and seal the annular gap optimally.

It is advantageous if the sealing element is configured as a stand-alone component. In particular, this simplifies a manufacturing process of the instrument. The sealing element then only needs to be inserted into the annular gap when the instrument is assembled. For example, the sealing element can thus be configured in a simple manner as an injection molded part. In particular, it is favorable if the sealing element is releasably connectable to the connecting element and the first instrument part. This makes it possible, in particular, to replace a sealing element that may be damaged. For example, it is conceivable that an elastic sealing element, after being reprocessed multiple times, no longer has the required elasticity to completely or partially fill and to seal the annular gap in the desired manner. In this configuration, it can then be easily replaced.

Preferably, the sealing element is connected to the connecting element in a force-locking and/or positive-locking manner. Such a connection can be realized, in particular, by way of a clamping connection or a snapping connection. A force-locking and/or positive-locking connection of the sealing element to the connecting element can be realized, in particular, without additional tools and thus simplifies a manufacturing process of the instrument.

It is advantageous if the connecting element has an undercut relative to the longitudinal axis, into which undercut the sealing element engages. This may be the case, in particular in the assembly position. The undercut makes it possible, in particular, to form a positive-locking connection between the sealing element and the connecting element in order to thus hold the sealing element in the annular gap permanently and reliably, even when the instrument parts are pivoted relative to one another.

It is favorable if the sealing element is molded onto the at least one first annular face or onto the at least one second annular face or is materially bonded thereto. This can be achieved, in particular, by overmolding, adhesive bonding, or vulcanization. Such a configuration makes it possible, in particular, to simplify an assembly of the instrument, since the sealing element is then either already arranged on the first instrument part or on the connecting element when the instrument parts are connected to one another with the connecting element.

In accordance with a further preferred embodiment, provision may be made that formed on the first annular face and/or on the second annular face is at least one recess that is open pointing toward the respective other annular face, and that the sealing element engages at least partially, in particular completely, into the at least one recess in the assembly position. In particular, a good connection between the sealing element and the connecting element and/or the first instrument part can thus be established. In particular, a partial or complete positive engagement can thus be made possible. This allows the sealing element to be held in the desired position in the annular gap in a simple and secure manner.

In order to simplify the assembly of the sealing element, it is advantageous if the at least one recess is of circumferential configuration relative to the longitudinal axis. In particular, it may be configured in the form of an annular groove.

It is advantageous if the sealing element comprises at least one projection and if the at least one projection engages into the at least one recess in the assembly position. In particular, the engagement may be positive-locking. This enables, in particular, a permanent and secure connection of the sealing element with the connecting element and/or the first instrument part. In particular, the at least one projection may be formed on the sealing element only through the introduction into the annular gap in the case of an interference fit, such that part of the sealing element is able to dip into or be pressed into the at least one first and/or at least one second recess by deforming the sealing element.

The sealing element can be formed in a simple manner if the at least one projection is configured in the form of an annular flange pointing in the direction toward at least one of the two annular faces.

Preferably, the sealing element defines at least one outer annular face tapering in the direction toward the at least one second instrument part. This tapering outer annular face may be provided, in particular, in the basic position. It facilitates the insertion of the sealing element into the annular gap with interference when connecting the instrument parts to one another with the connecting element. The tapering outer annular face may, in particular, be conically shaped.

It is advantageous if the sealing element defines at least one outer annular face, which, in a longitudinal section view, is convexly curved pointing away from the longitudinal axis. In particular, said outer annular face may be formed in the basic position as described. Such an annular face allows the sealing element, in particular, to nestle into a recess in the second annular face on the first instrument part.

It is advantageous if the sealing element defines at least one cylindrical outer annular face pointing away from the longitudinal axis. In particular, it may have such an annular face in the basic position. Such an annular face can be produced in a simple manner. In particular, it is advantageous if it extends up to an end of the sealing element pointing away from the at least one second instrument part. Thus, the sealing element can fill the annular gap, in particular completely, up to a side face of the first instrument part pointing away from the at least one second instrument part.

In order to simplify the insertion of the sealing element into the annular gap when assembling the instrument, it is favorable if an outer diameter of the sealing element, in particular in the basic position, is smaller at an end pointing toward the at least one second instrument part than at an end pointing away from the at least one second instrument part.

In accordance with a further preferred embodiment, provision may be made that a through-bore is formed on the first instrument part concentrically to the longitudinal axis, that the head recess is configured in the form of a receiving portion of the through-bore that is expanded in inner diameter, and that the head recess extends commencing from a first end of the through-bore, which points away from the at least one second instrument part, in the direction toward the at least one second instrument part. In this way, the first instrument part can be easily prepared for receiving the connecting element, in particular its head in the head recess.

The head recess can be formed in a simple manner if the receiving portion forms a first hollow-cylindrical bore portion. It can then be formed in a simple manner, for example, with a twist drill defining a cylindrical outer face.

A stop for a head of the connecting element can be formed on the first instrument part in a simple manner if the through-bore comprises a bore portion that adjoins the receiving portion and tapers in the direction toward the at least one second instrument part. In particular, the bore portion may taper conically. It can be formed in a simple manner, for example, with a countersink drill.

It is favorable if a countersink is formed on the head recess commencing from the first end. It may be configured, in particular, in the form of a countersink portion that conically expands pointing away from the at least one second instrument part. Such a countersink portion can be formed in a simple manner with a countersink drill. In particular, it facilitates the insertion of the sealing element into the annular gap during the assembly of the instrument.

Preferably, the through-bore comprises a second hollow-cylindrical bore portion commencing from a second end pointing toward the at least one second instrument part in the direction toward the first end. It can serve, in particular, to allow a connecting portion of the connecting element to pass therethrough in order to bring said connecting portion into engagement with the at least one second instrument part. In addition, such a hollow-cylindrical bore portion can be formed in a simple manner with a cylindrical drill.

It is favorable if a first inner diameter of the first hollow-cylindrical bore portion is greater than a second inner diameter of the second hollow-cylindrical bore portion. In this way, in particular, it can be prevented that the head of the connecting element is able to pass through the through-bore.

In the assembly position, the sealing element preferably extends at least up to the first end of the through-bore. In particular, a flush fitting of the sealing element in the annular gap can be made possible, thereby conveying a clean appearance of the instrument. In addition, any protruding edges of the sealing element in the region of the annular gap that protrude from an outer face of the first instrument part can be avoided.

Two instrument parts can be connected to one another in a simple manner if the connecting element is configured in the form of a screw or in the form of a rivet.

In order to delimit a movement of the sealing element in the direction toward the at least one second instrument part, it is advantageous if the head comprises an annular stop face, which surrounds the longitudinal axis, extends transversely to the longitudinal axis, and points away from the at least one second instrument part.

For a good sealing of the annular gap, it is favorable if the sealing element in the assembly position is in contact with the stop face.

Good haptics of the instrument can be achieved, in particular, in that the end of the sealing element pointing away from the at least one second instrument part, in the assembly position, is flush with an outer surface of the first instrument part. This outer surface of the instrument part can then be formed completely smooth and flat, even in the region of the through-bore, which is filled by the head of the connecting element and the sealing element.

In accordance with a further preferred embodiment, provision may be made that a second self-contained sealing element surrounding the longitudinal axis is arranged or formed between the first instrument part and the at least one second instrument part, and that the second sealing element is in contact with the first and the at least one second instrument part, in particular with mutually facing side faces thereof. A second sealing element arranged and formed in that way can prevent, in particular, body fluids or cleaning fluids that penetrate between the mutually facing side faces of the instrument parts from being able to travel into the annular gap due to capillary action, namely commencing from an end of the through-bore that points in the direction toward that at least one second instrument part. In conjunction with the sealing element inserted in the annular gap, the annular gap can thus be closed completely, so that it is possible to clean the instrument in a simpler and more reliable manner.

Preferably, the first instrument part and/or the second instrument part comprise a sealing element receptacle for the second sealing element. It can thus be positioned on the first and/or second instrument part in a defined manner.

The sealing element receptacle can be formed in a simple manner if it is configured in the form of an annular groove that concentrically surrounds the longitudinal axis and is open pointing in the direction toward the respective other instrument part. For example, an annular seal can be inserted into such an annular groove.

In order to achieve a secure seal, it is advantageous if the second sealing element and the sealing element receptacle form an interference fit.

In particular, the instrument can be produced and assembled in a simple manner if the second sealing element is configured in the form of a self-contained sealing ring. In particular, the sealing ring may have a circular cross section in a basic position.

The two instrument parts can be coupled to one another in a simple manner if the connecting element comprises a connecting portion projecting from the head and pointing in the direction toward the at least one second instrument part. Such a connecting portion can, for example, be brought into force-locking and/or positive-locking engagement with the at least one second instrument part in order to couple the instrument parts of the instrument to one another so as to be movable in the desired manner.

It is favorable if a connecting portion receptacle is formed on the at least one second instrument part and if the connecting portion and the connecting portion receptacle are in force-locking and/or positive-locking engagement in the assembly position. Such a connecting portion receptacle enables, in particular, a simple connection to the connecting portion of the connecting element. The connecting portion receptacle may be configured, in particular, in the form of a connecting bore. For a connection of the instrument parts with a rivet, the connecting receptacle does not necessarily have to be provided with an internal thread.

It is advantageous if an external thread is formed on the connecting portion and if an internal thread corresponding to the external thread is formed on the connecting portion receptacle. This makes it possible, in particular, to screw the connecting element in the form of a screw with the connecting portion into the connecting portion receptacle. The first instrument part is then movably held between the head of the connecting element and the at least one second instrument part.

Preferably, the sealing element is made of an elastically and/or plastically deformable plastic material. This also applies to the second sealing element. In particular, interference fits can be realized in a simple manner with such plastic materials, in order to securely and permanently seal or fill the annular gap.

It is advantageous if the plastic material is or contains a natural rubber, an elastoplastic, an elastomer, in particular a thermoplastic elastomer, in particular acrylonitrile-butadiene rubber, styrene-butadiene rubber, hydrogenated acrylonitrile-butadiene rubber, chloroprene rubber, acrylate rubber, ethylene acrylate rubber, fluororubber, perfluororubber, silicone rubber, fluoro-silicone rubber, polyurethane rubber, ethylene-propylene rubber, epichlorohydrin rubber, natural rubber, polyisoprene rubber. Such plastic materials have the required properties for use in sealing the annular gap.

Preferably, the plastic material is sterilizable with superheated steam. This has the advantage that the medical instrument can be reprocessed in the desired manner.

In order to achieve a secure and permanent sealing effect, it is favorable if the sealing element is formed in one piece, in particular monolithically.

The object stated at the outset is further achieved, in accordance with the disclosure, in a medical instrument set of the kind described at the outset in that the at least two instruments are configured in the form of one of the medical instruments described above.

The medical instrument set then has the advantages that have already been described above in connection with preferred embodiments of medical instruments.

It is advantageous if the sealing elements of the at least two different medical instruments are differently colored. Thus, the sealing element, which fills the annular gap at least partially, can be used in a simple manner to color-code the instruments. Different properties, such as different shapes or types of instruments, can then be coded with corresponding colors. For example, the sealing elements may be blue, red, yellow, green, black, white, gray, or brown.

### BRIEF DESCRIPTION OF THE DRAWING FIGURES

The subsequent description of preferred embodiments of the disclosure serves in conjunction with the drawings for further explanation.

FIG. 1 shows a schematic perspective total view of an embodiment of a medical instrument;

FIG. 2 shows a schematic enlarged view of the region A in FIG. 1 in an exploded depiction;

FIG. 3 shows a section view along line 3-3 in FIG. 2;

FIG. 4 shows a section view analogous to FIG. 3 with a sealing element inserted in the annular gap;

FIG. 5 shows a view analogous to FIG. 3 of a further embodiment;

FIG. 6 shows a view analogous to FIG. 3 of a further embodiment;

FIG. 7 shows schematic depictions of connecting elements and differently colored sealing elements;

FIG. 8 shows a section view analogous to FIG. 3 of a further embodiment;

FIG. 9 shows a section view of a further embodiment of a sealing element;

FIG. 10 shows a section view depiction of a further embodiment of a sealing element; and

FIG. 11 shows a schematic section view of a further embodiment of a connecting element.

### DETAILED DESCRIPTION

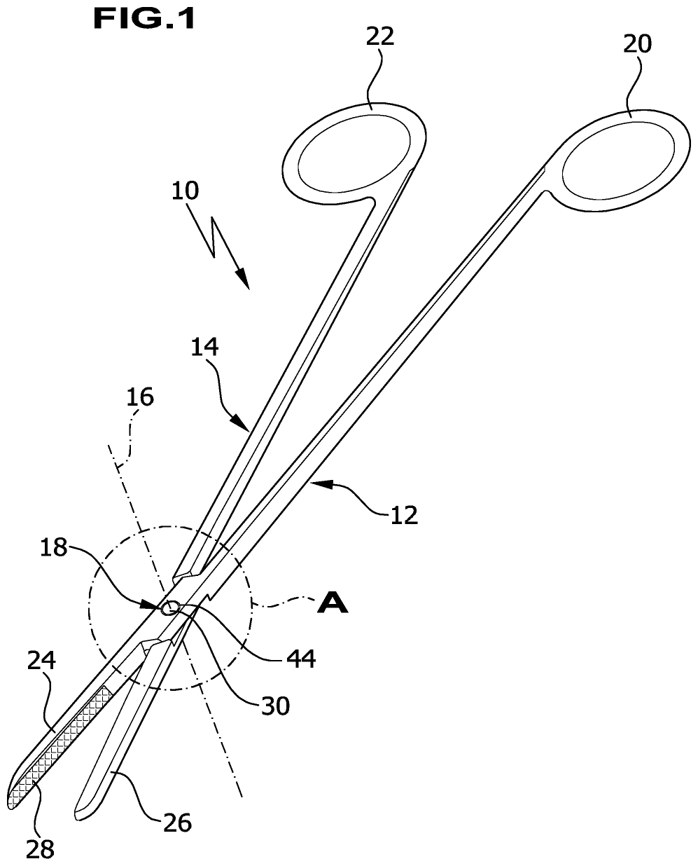

An embodiment of a medical instrument 10 is schematically depicted in FIG. 1. It comprises a first instrument part 12 and a second instrument part 14. These are mounted to one another so as to be pivotable about a pivot axis 16 and are coupled to one another with a connecting element 18.

The two instrument parts 12 and 14 are configured in the form of elongate branches, at each proximal end of which a respective finger ring 20 and 22 is arranged. On the distal side of the pivot axis 16, cooperating tool elements 24 and 26 are arranged or formed on the instrument parts 12 and 14. In the embodiment of FIG. 1, these have mutually facing clamping faces 28, so that the instrument 10 can be used as a clamp.

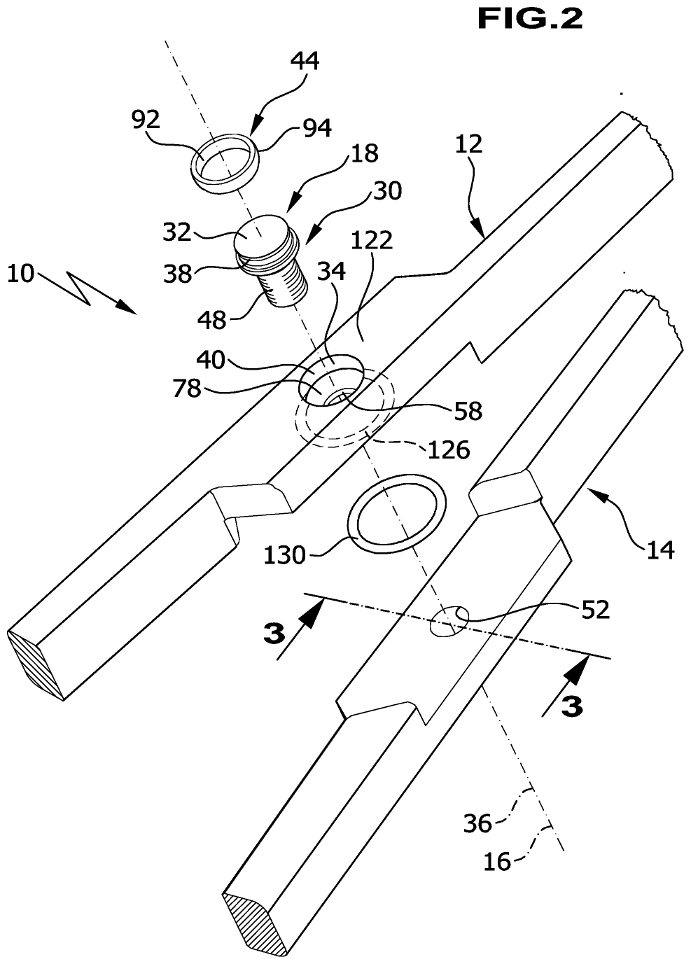

In the embodiment schematically depicted in FIG. 2, the connecting element 18 is configured in the form of a connecting screw 30 and comprises a head 32. The head 32 is accommodated in a head recess 34 on the first instrument part 12. It is open pointing away from the first instrument part 12 in a direction away from the second instrument part 14.

The head 32 has a circumferential, self-contained first annular face 38 pointing away from a longitudinal axis 36 of the connecting element 18. Furthermore, the head recess 34 has a circumferential, self-contained second annular face 40 pointing in the direction toward the longitudinal axis 36. The two annular faces 38 and 40 are separated from one another in the radial direction relative to the longitudinal axis 36 by an annular gap 42.

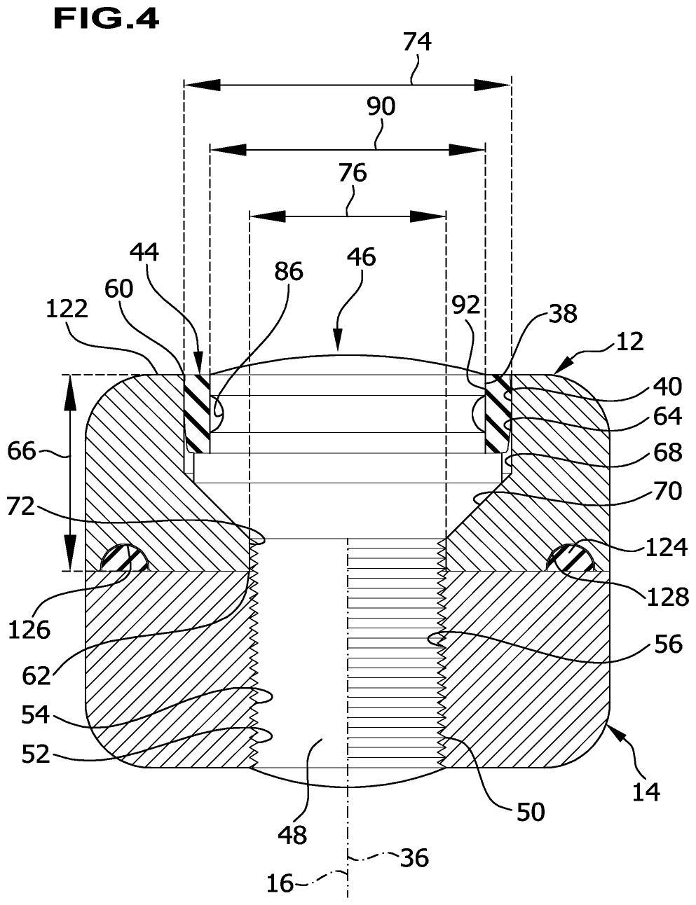

A sealing element 44 is arranged in the annular gap 42. As schematically depicted in FIG. 4 in an assembly position, it is in contact with both annular faces 38 and 40.

The structure of a connection region of the medical instrument 10 according to the embodiment schematically depicted in FIGS. 2 to 4 is described in detail in the following.

In this embodiment, the connecting element 18 is configured, as already mentioned, in the form of a screw 46. A connecting portion 48 projects from the head 32 of the connecting element 18 pointing in the direction toward the second instrument part 14. Said connecting portion 48 is provided with an external thread 50.

A connecting portion receptacle 52 is formed on the second instrument part 14, namely in the form of a connecting bore 54. An internal thread 56 corresponding to the external thread 50 is formed on the connecting portion receptacle 52.

The described configuration of the connecting element 18 allows the connecting portion 48 to be screwed to the connecting portion receptacle 52. Thus, the connecting portion 48 and the connecting portion receptacle 52 are in engagement in a force-locking and/or positive-locking manner in an assembly position, in which the instrument parts 12 and 14 are movably coupled to one another with the connecting element 18.

Furthermore, a through-bore 58 is formed on the first instrument part 12 concentrically to the longitudinal axis 36 defining the pivot axis 16. The through-bore 58 has a first end 60 and a second end 62. The first end 60 points away from the second instrument part 14. The second end 62 points toward the second instrument part 14.

The head recess 34 is configured in the form of a receiving portion 64 of the through-bore 58 that is expanded in inner diameter. It extends from the first end 60 in the direction toward the second instrument part 14, namely in the direction of the longitudinal axis 36 on about half a thickness 66 of the first instrument part 12. The receiving portion 64 is formed by a first hollow-cylindrical bore portion 68.

The receiving portion 64 is adjoined by a conically tapering bore portion 70 in the direction toward the second instrument part 14.

The through-bore 58 further comprises a second hollow-cylindrical bore portion 72. Said bore portion 72 extends from the conically tapering bore portion 70 up to the second end 62. A first inner diameter 74 of the first hollow-cylindrical bore portion 68 is greater than a second inner diameter 76 of the second hollow-cylindrical bore portion 72, namely slightly more than 1.5 times greater.

The head of the connecting element 18 conically expands in diameter commencing from the connecting portion 48 up to a short cylindrical portion 80 extending in the direction of the longitudinal axis 36. The first annular face 38 is offset somewhat in the direction toward the longitudinal axis 36 relative to the portion 80, so that an annular stop face 82 pointing away from the second instrument part 14 is formed. The stop face on the head 32 surrounds the longitudinal axis 36 and extends transversely, namely perpendicularly, thereto.

A recess 84 that is open toward the second annular face 40 is formed on the first annular face 38. It is of circumferential configuration relative to the longitudinal axis 36, namely in the form of an annular groove 86 and, in a longitudinal section view, is concavely curved pointing away from the longitudinal axis 36.

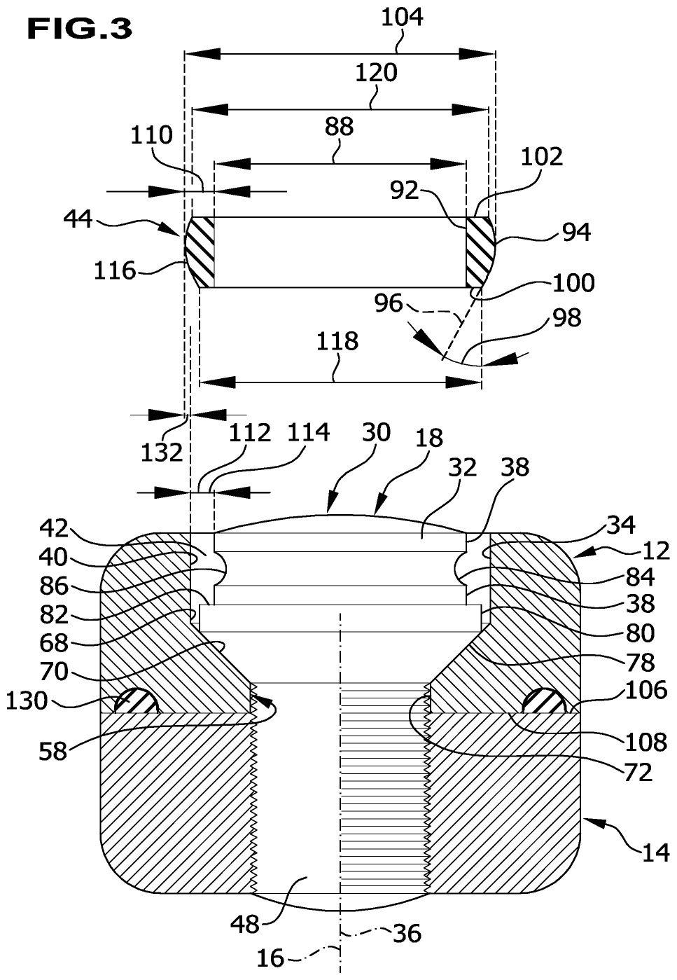

In FIGS. 2 and 3, the sealing element 44 is schematically depicted in a basic position, in which it is completely separated from the connecting element 18. The sealing element 44 adopts the basic position after its production. It is formed separately from the connecting element 18.

The sealing element 44 is configured in the form of a short sleeve extending in the direction of the longitudinal axis 26 and defines an inner diameter 88, which corresponds to an outer diameter 90 of the head 32 in the region of the first annular face 38. An inner wall face 92 of the sealing element 44 pointing in the direction toward the longitudinal axis 36 is configured in the form of a hollow-cylindrical wall face.

An outer face 94 that points away from the longitudinal axis 36 is convexly curved. A tangent 96 to the outer face 94 in the region of an end pointing toward the second instrument part 14 encloses an angle 98 with the longitudinal axis 36, which angle is in a range between 20° and 40°.

A first end face 100 of the sealing element 44, which points in the direction toward the second instrument part 14, is configured in the form of a planar annular face extending perpendicularly to the longitudinal axis 36. A second end face 102 of the sealing element 44 is configured pointing away from the second instrument part 14 in the form of a planar annular face extending perpendicularly to the longitudinal axis 36.

A maximum outer diameter 104 of the sealing element 44 is slightly larger than the first inner diameter 74 of the receiving portion 64. The sealing element 44 is made of an elastic plastic material. This allows the sealing element 44 to be pushed over the head 32, the wall face 92 abutting against the first annular face 38, namely so far until the second end face 102 strikes the stop face 82.

The connecting element 18 prepared for the assembly of the instrument 10 in this way can now be screwed leading with the connecting portion 48 into the first end 60 of the through-bore 58 and then into the connecting portion receptacle 52 until the conically expanding portion 78 and the conically tapering bore portion 70 come into abutment. When the connecting element 18 is further screwed to the second instrument part 14, said connecting element is drawn with a side face 106 pointing in the direction toward the first instrument part 12 against a side face 108 of the first instrument part 12 pointing in the direction toward the second instrument part 14.

When screwing in the connecting element 18, the curved outer face 94 allows easy insertion of the sealing element 44 into the annular gap 42. The sealing element 44 is hereby slightly compressed. Starting from its original thickness 110 in the basic position, it is compressed in the assembly position to a thickness 112, which corresponds to a distance 114 of the annular faces 38 and 40 from one another. Thus, an interference fit is formed between the sealing element 44 and the annular gap 42.

The described compression of the sealing element 44 has the result that in the basic position, in which it is out of engagement with the annular faces 38 and 40, it defines a first sealing element shape, that in the assembly position, in which it is arranged in the annular gap 42, it defines a second sealing element shape, and that the second sealing element shape and the first sealing element shape differ from one another.

In this embodiment, the sealing element 44 is configured, as described, as a stand-alone component and is releasably connectable to the connecting element 18 and the first instrument part 12. Furthermore, as described, it is connected to the connecting element in a force-locking and/or positive-locking manner, namely by way of a clamping connection in the case of the embodiment in FIGS. 3 and 4.

The sealing element 44 thus has, in the longitudinal section view as shown schematically in FIG. 3, a convex curved outer annular face 116 pointing away from the longitudinal axis 36, which annular face is defined by the outer face 94 in the basic position. Furthermore, an outer diameter 118 of the sealing element 44 in the basic position in the transition to the first end face 100 is smaller than an outer diameter 120 in the transition to the second end face 102.

In the assembly position as schematically depicted in FIG. 4, the second end face 102 of the sealing element 44 is flush with an outer surface 122 of the first instrument part 12.

The embodiment of FIGS. 3 and 4 comprises a further sealing element 124. This can also optionally be omitted.

The sealing element 124 is arranged between the first instrument part 12 and the second instrument part 14, is self-contained, and surrounds the longitudinal axis 36. It is in contact with both instrument parts 12 and 14, namely with the mutually facing side faces 106 and 108.

In the embodiment of FIGS. 3 and 4, a sealing element receptacle 126 for the second sealing element 124 is formed on the first instrument part 12. It is configured in the form of an annular groove 128 that concentrically surrounds the longitudinal axis 36 and is open pointing in the direction toward the second instrument part 14.

The second sealing element 124 is configured in the form of a self-contained sealing ring 130, which is circular in cross section. In order to achieve a good seal between the two instrument parts 12 and 14, the second sealing element 124 and the sealing element receptacle 126 also form an interference fit. In other words, the second sealing element 124 deforms during the assembly of the instrument, so that it nestles into the sealing element receptacle 126 on the one hand and is in surface-to-surface contact with the side face 106 of the second instrument part on the other hand.

Both sealing elements 44 and 124 are formed in one piece, namely monolithically.

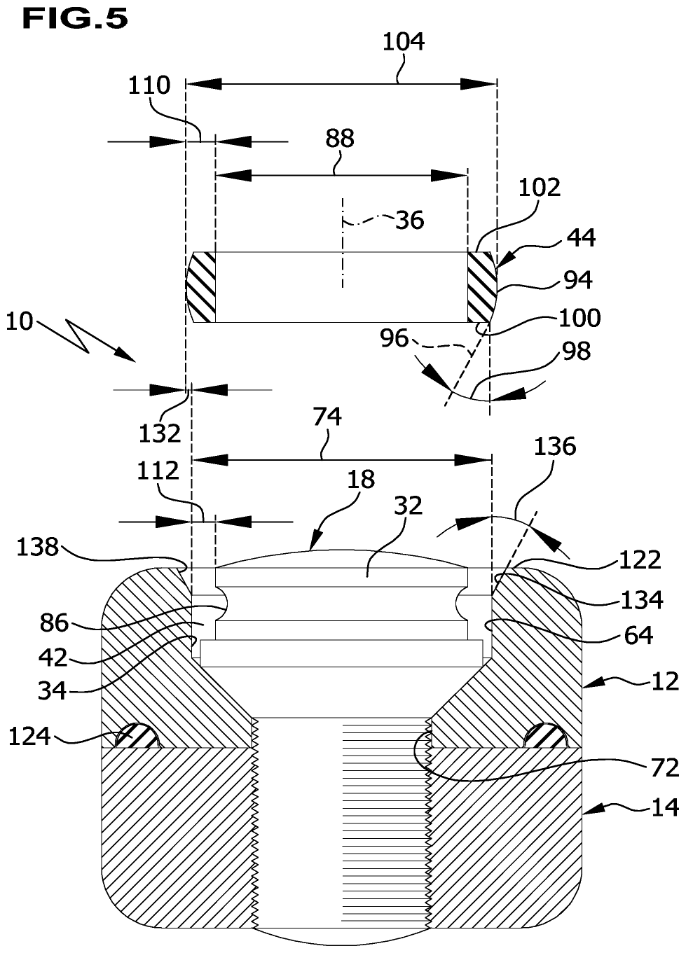

A further embodiment of a medical instrument 10 is schematically depicted in FIG. 5 in section. It corresponds substantially to the arrangement depicted in FIG. 3, such that the same reference numerals are used here for identical or functionally similar components as in the embodiment of FIGS. 2 to 4.

A first difference lies in the design of the sealing element 44. In this case, the end faces 100 and 102 are identical, whereas the end face 100 in the embodiment of FIG. 3 is narrower than the end face 102. However, the outer face 94 is convexly curved pointing away from the longitudinal axis 36 in the embodiment of FIG. 5, too.

In this embodiment, too, there is an interference 132 of half the difference between the maximum outer diameter 104 of the sealing element 44 in the basic position and the first inner diameter 74 of the receiving portion 64.

In order to facilitate the insertion of the connecting element with the sealing element 44 arranged on the head 32 into the head recess 34, a countersink 134 is formed commencing from the surface 122 of the first instrument part 12. A countersink angle 136 between a countersink surface 138 that is defined by the countersink 134 and conically tapers in the direction toward the second instrument part 14 and the longitudinal axis 36 is in a range of about 20° to about 30°.

The countersink 134 in cooperation with the convexly curved outer face 94, which, as described in the embodiment of FIG. 3, defines an angle 98 between a tangent 96 to the outer face 94 with the longitudinal axis 36, facilitates the deformation of the sealing element 44 that has an interference 132 in the basic position.

In the embodiment of FIG. 5, too, a second sealing element 124 is schematically provided and depicted. Here, too, the second sealing element 124 may optionally be omitted in an embodiment that is not depicted.

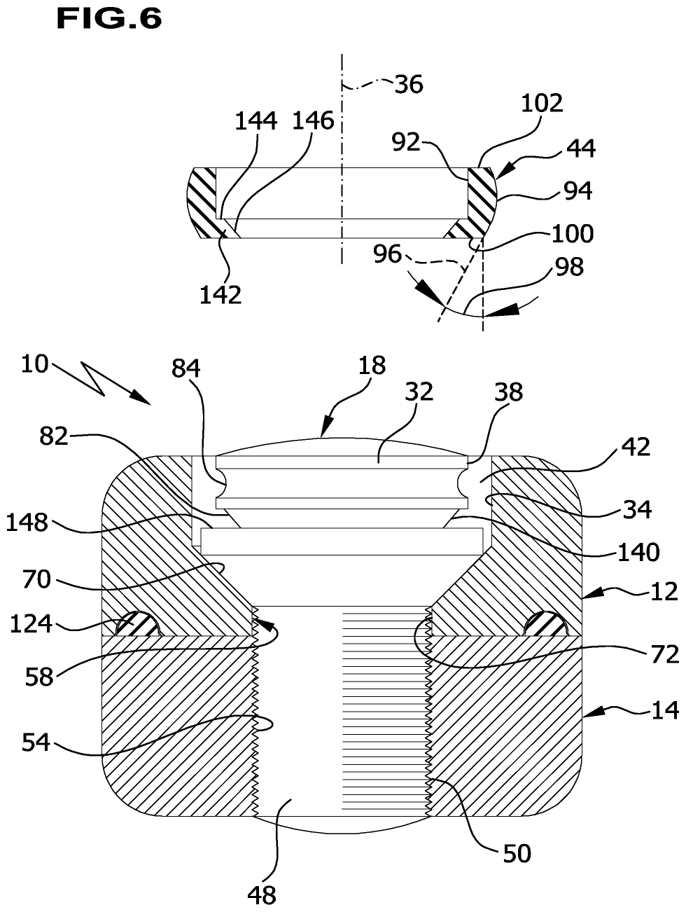

A further embodiment of a medical instrument 10 is schematically depicted in FIG. 6 in a section view analogous to FIG. 3. It differs from the arrangement according to FIG. 3 in the design of the sealing element 44 and the head 32 of the connecting element 18. Only the differences in the embodiments are discussed in the following. In all other respects, reference is made to the description of the embodiment depicted in FIG. 3.

At the head 32, the first annular face 38 is provided with an undercut 140 directly adjoining the stop face 82, which undercut serves to accommodate in a positive-locking manner an annular flange 142 on the sealing element 44 projecting in the direction toward the longitudinal axis 36. The flange 142 is delimited on the one hand by the first end face 100 and on the other hand by an annular face 144 that extends in parallel to said end face 100 and points away from the second instrument part 14.

A front edge 146 of the flange 142 tapers conically from the annular face 144 to the first end face 100. Accordingly, a corresponding conical face 148 is formed on the undercut 140. In the embodiment depicted in FIG. 6, the sealing element 44 can thus be connected to the connecting element 18 in a force-locking and/or positive-locking manner by way of a clamping connection or a snapping connection.

The sealing element 44 can be used to identify medical instruments 10. Different types or sizes of instruments 10 can be coded, for example, with differently colored sealing elements.

FIG. 7 shows three different sealing elements 44, which are colored differently. They can optionally be connected to the connecting element 18.

Of course, the sealing elements 44 of the embodiments described above may also be provided in any colors in order to identify or code the respective instrument 10 in the desired manner.

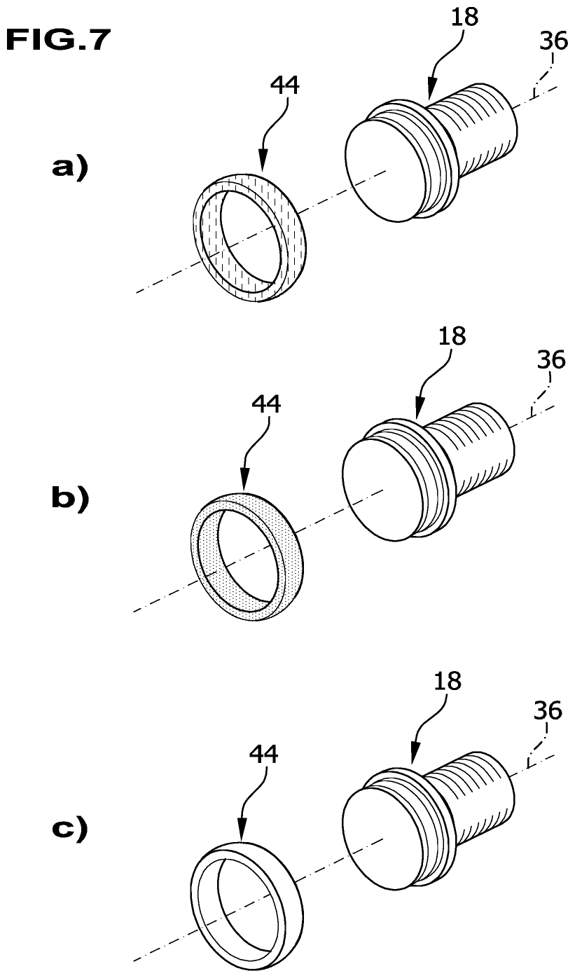

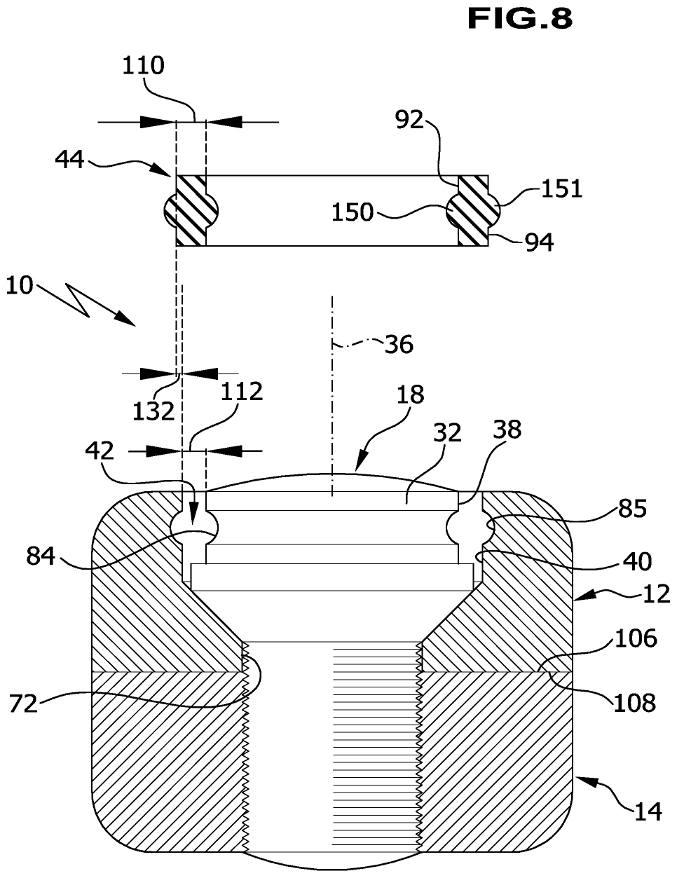

FIG. 8 schematically shows a further embodiment of a medical instrument in a section view analogous to FIG. 3. Here, too, only the differences to the embodiment of FIG. 3 are discussed in the following.

In the embodiment of FIG. 8, no second sealing element 124 is provided. In this form, as already mentioned above, the second sealing element 124 can in principle be omitted in the preceding embodiments, too.

The sealing element 44 has a cylindrical sleeve shape. The wall face 92 and the outer face 94 are formed concentrically to one another. A projection 150 projects from the wall face 92 in the direction toward the longitudinal axis 36. The projection 150 is of self-contained annular configuration. In an analogous manner, a projection 151 projects from the outer face 94. It points in the radial direction away from the longitudinal axis 36 and is also of annular, self-contained configuration.

The projection 150 is formed correspondingly to the recess 84 on the head 32 of the connecting element 18. Opposite the recess 84, a recess 85 that corresponds to the projection 151 is formed on the second annular face 40. The recesses 84, 85 are dimensioned such that the projections 150 and 151 are received in a positive-locking manner when the sealing element 44, which here, too, is formed with interference 132, is accommodated in the annular gap 42.

In this embodiment, the sealing element 44 defines a cylindrical outer annular face pointing away from the longitudinal axis 36, namely through the outer face 94.

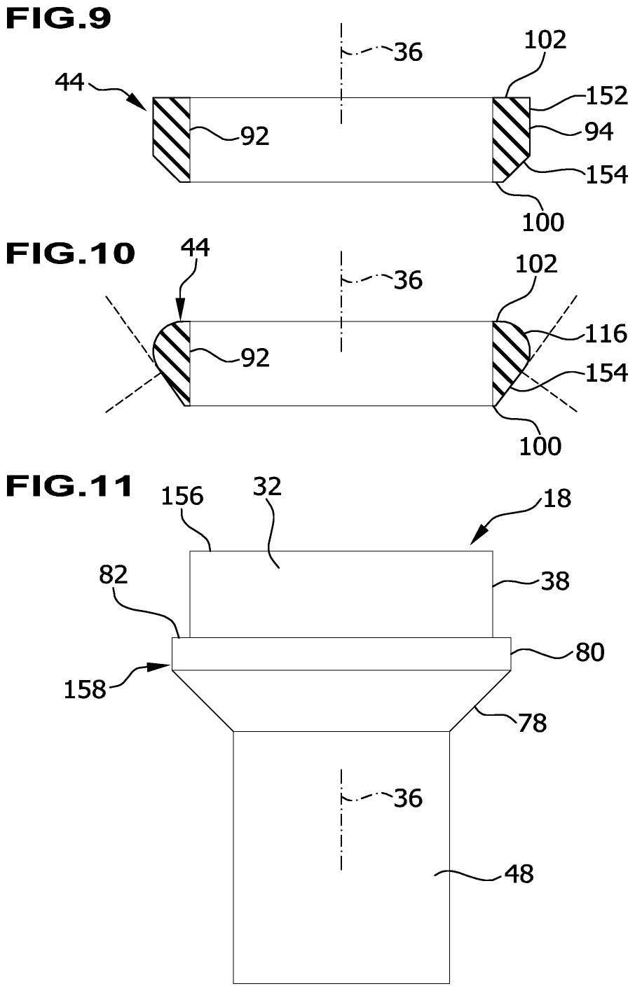

A further embodiment of a sealing element 44 is schematically depicted in FIG. 9. The inner wall face 92 extends concentrically to the longitudinal axis 36. Commencing from the second end face 102, a portion of the outer face 94 that defines a cylindrical outer annular face 152 extends in the direction toward the first end face 100. This cylindrical annular face 152 is adjoined in the direction of the first end face 100 by a conically tapering outer annular face 154.

A further embodiment of a sealing element is schematically depicted in FIG. 10. It differs from the embodiment of FIG. 9 in that instead of the cylindrical outer annular face 152, an outer annular face 116 which is convex curved pointing away from the longitudinal axis 36 is formed.

The embodiments of the sealing elements of FIGS. 9 and 10 can be combined, in particular, with all the connecting elements 18 already described above.

Furthermore, they can also be combined with a connecting element 18 schematically depicted in FIG. 11. Said connecting element 18 has a planar end face 156, which points away from the connecting portion 48. The connecting portion 48, in turn, is adjoined by a conically expanding portion 78, which transitions into a short cylindrical portion 80. Said cylindrical portion 80 is adjoined by the annular stop face 82 pointing away from the connecting portion 78. Unlike in the embodiments described above, the first annular face 38 is configured without a recess 84.

The embodiment depicted schematically in FIG. 11 is configured as a rivet 158.

In a further embodiment, which corresponds schematically to the arrangement in FIG. 11, an external thread 50 is formed on the connecting portion 48, so that this variant can be used as a screw 46.

In embodiments not shown, the sealing elements 44 described above are molded onto the first annular face 38 or are materially bonded thereto by overmolding, adhesive bonding, or vulcanization.

In further embodiments, the respective sealing element 44 is molded onto the second annular face 40 or materially bonded thereto, namely by overmolding, adhesive bonding, or vulcanization.

In both cases, a secure connection between the sealing element 44 and the connecting element 18 on the one hand or between the sealing element 44 and the first instrument part 12 on the other hand can be established.

As already explained, all sealing elements 44 described above are made of an elastically and/or plastically deformable plastic material.

The plastic material is or contains a natural rubber, an elastoplastic, an elastomer, in particular a thermoplastic elastomer. In particular, the plastic is acrylonitrile-butadiene rubber, styrene-butadiene rubber, hydrogenated acrylonitrile-butadiene rubber, chloroprene rubber, acrylate rubber, ethylene acrylate rubber, fluororubber, perfluororubber, silicone rubber, fluoro-silicone rubber, polyurethane rubber, ethylene-propylene rubber, epichlorohydrin rubber, natural rubber, or polyisoprene rubber.

In order to enable the processing of the medical instruments 10 in a dishwasher and in a superheated steam sterilizer, the plastic material used for forming the sealing element 44 is sterilizable with superheated steam.

The described embodiments of medical instruments 10, due to the sealing element 44 inserted in the annular gap 42 between the head 32 of the connecting element 18 and the receiving portion 64, prevent the ingress of rinsing liquids, body fluids, or other contaminants into the annular gap 42. Thus, the cleanability of the instrument 10 can be improved, since the risk of contamination of the annular gap 42 is significantly reduced.

1. A medical instrument comprising:
a first instrument part; and
at least one second instrument part,
the first instrument part and the at least one second instrument part being mounted to one another so as to be pivotable about a pivot axis and coupled to one another with a connecting element,
a longitudinal axis of the connecting element defining the pivot axis,
the connecting element having a head at least partially accommodated in a head recess on the first instrument part,
the head recess being open pointing from the first instrument part in a direction away from the at least one second instrument part,
the head having at least one first annular face that is circumferential, self-contained, and pointing away from the longitudinal axis,
the head recess having at least one second annular face that is circumferential, self-contained, and pointing toward the longitudinal axis,
the at least one first annular face and the at least one second annular face being separated from one another in a radial direction relative to the longitudinal axis by an annular gap, and
a sealing element being arranged in the annular gap, the sealing element being in contact with the at least one first annular face and with the at least one second annular face.

2. The medical instrument according to claim 1, wherein the sealing element and the annular gap form an interference fit.

3. The medical instrument according to claim 1, wherein the sealing element:
a) in a basic position, in which the sealing element is out of engagement with the first annular face and/or the second annular face, defines a first sealing element shape, and wherein the sealing element, in an assembly position, in which the sealing element is arranged in the annular gap, defines a second sealing element shape, the second sealing element shape differing from the first sealing element shape,
and/or
b) is configured as a stand-alone component,
and/or
c) is connected to the connecting element in a force-locking and/or positive-locking manner.

4. The medical instrument according to claim 1, wherein the connecting element has an undercut relative to the longitudinal axis, into which undercut the sealing element engages.

5. The medical instrument according to claim 1, wherein the sealing element is molded onto or is materially bonded to the at least one first annular face or the at least one second annular face.

6. The medical instrument according to claim 1, wherein, formed on the first annular face and/or on the second annular face is at least one recess that is open pointing toward the respective other annular face, and wherein the sealing element engages at least partially into the at least one recess in the assembly position.

7. The medical instrument according to claim 1, wherein:
a) the sealing element defines at least one outer annular face that tapers toward the at least one second instrument part,
and/or
b) the sealing element defines at least one outer annular face that, in a longitudinal section view, is convexly curved pointing away from the longitudinal axis,
and/or
c) the sealing element defines at least one cylindrical outer annular face pointing away from the longitudinal axis,
and/or
d) an outer diameter of the sealing element is smaller at an end pointing toward the at least one second instrument part than at an end pointing away from the at least one second instrument part.

8. The medical instrument according to claim 1, wherein a through-bore is formed on the first instrument part concentrically to the longitudinal axis, wherein the head recess is configured as a receiving portion of the through-bore that is expanded in inner diameter, and wherein the head recess extends commencing from a first end of the through-bore, which points away from the at least one second instrument part, toward the at least one second instrument part.

9. The medical instrument according to claim 8, wherein:
a) the receiving portion forms a first hollow-cylindrical bore portion,
and/or
a) the through-bore comprises a bore portion, which adjoins the receiving portion and tapers toward the at least one second instrument part,
and/or
c) a countersink is formed on the head recess commencing from the first end,
and/or
d) the through-bore comprises a second hollow-cylindrical bore portion commencing from a second end pointing toward the at least one second instrument part toward the first end, wherein a first inner diameter of the first hollow-cylindrical bore portion is greater than a second inner diameter of the second hollow-cylindrical bore portion,
and/or
e) the sealing element in the assembly position extends at least up to the first end of the through-bore.

10. The medical instrument according to claim 1, wherein the connecting element is configured as a screw or a rivet.

11. The medical instrument according to claim 1, wherein the head comprises an annular stop face, which surrounds the longitudinal axis, extends transversely to the longitudinal axis, and points away from the at least one second instrument part.

12. The medical instrument according to claim 1, wherein the end of the sealing element pointing away from the at least one second instrument part, in the assembly position, is flush with an outer surface of the first instrument part.

13. The medical instrument according to claim 1, wherein a second self-contained sealing element surrounding the longitudinal axis is arranged or formed between the first instrument part and the at least one second instrument part, and the second sealing element is in contact with the first instrument part and the at least one second instrument part.

14. The medical instrument according to claim 13, wherein:
a) the first instrument part and/or the second instrument part comprise(s) a sealing element receptacle for the second sealing element,
and/or
b) the second sealing element and the sealing element receptacle form an interference fit,
and/or
c) the second sealing element is configured as a self-contained sealing ring.

15. The medical instrument according to claim 1, wherein:
the connecting element comprises a connecting portion projecting from the head and pointing toward the at least one second instrument part,
the connecting portion and the connecting portion receptacle are in force-locking and/or positive-locking engagement in the assembly position, and
an internal thread corresponding to the external thread is formed on the connecting portion receptacle.

16. The medical instrument according to claim 1, wherein the sealing element is made of an elastically and/or plastically deformable plastic material.

17. The medical instrument according to claim 1, wherein the sealing element is formed in one piece.

18. A medical instrument set comprising at least two medical instruments according to claim 1. wherein the at least two medical instruments differ in shape. type, and/or size.

19. The medical instrument set according to claim 18, wherein the sealing elements of the at least two medical instruments are differently colored.
